(19) United States
(12) Patent Application Publication
(10) Pub. No.: US 2007/0105802 A1
(43) Pub. Date: May 10, 2007

(54) Compositions and methods for modulating and monitoring neurotransmitter receptor populations

(76) Inventors: James Eberwine, Philadelphia, PA (US); Janet Estee Kacharmina, Philadelphia, PA (US)

Correspondence Address: Jane Massey Licata;Licata & Tyrrell P.C., 66 E. Main Street, Marlton, NJ, 08053, US

(21) Appl. No.: 11/409,246

(22) Filed: Apr. 21, 2006

Related U.S. Application Data
(63) Continuation of application No. 10/673,070, filed on Sep. 26, 2003, now abandoned.
Application No. 10/673,070 is a continuation of application No. 09/464,270, filed on Dec. 17, 1999, now abandoned.

(51) Int. Cl. A61K 48/00

(52) U.S. Cl. 514/44

(57) ABSTRACT

Compositions and methods modulating neurotransmitter receptor populations are provided.

### INTRODUCTION

This application is a continuation of U.S. Ser. No. 10/673,070 filed Sep. 26, 2003, which is a continuation of U.S. Ser. No. 09/464,270 filed Dec. 17, 1999, which is herein incorporated by reference in its entirety. This invention was supported in part by funds from the U.S. government (NIH Grant No. AG09900-08 and MH158561-01) and the U.S. government may therefore have certain rights in the invention.

### BACKGROUND OF THE INVENTION

Many neuropsychiatric disorders and other disorders of the brain have been linked to a genetic mechanism. Although the genetic defects or differences may be diverse, the unifying theme has been brain development patterns and maintenance of neurological networks in the brain. Neurological networks are composed of many individual neurons, each neuron being a separate structural and functional cellular unit. Neurons in mammalian brain commonly have long cytoplasmic processes known as neurites which end in close apposition to other neighboring cells. The ends of the neurites are called synaptic terminals and the cell-to-cell contact is known as synapses. In higher animals, such as man, neurites are specialized to form dendrites and axons which conduct impulses to and from the cell body of the neuron. Therefore, neurons of the central nervous system of humans consist of discrete segments including the cell body, the dendrites, and the axon.

Excitatory synaptic transmission early in brain development is mediated by the actions of glutamate on NMDA receptors, since these synapses lack functional AMPA receptors. They are called electrophysiologically silent synapses since transmission is not detected at resting membrane potential. As brain development progresses, there is a conversion of the silent synaptic cleft to an active synaptic cleft (Petralia, R. S. et al. 1999. Nature Neurosci. 2:31-36), although a population of potentially silent synapses containing NMDA receptors but lacking AMPA receptor subunits is present in the adult hippocampus as well. Therefore, the conversion of silent to active synaptic junctions is in part dependent on the acquisition of functional AMPA receptors and is believed to be an important regulatory control of synaptic plasticity in the developing brain. Further, such a mechanism for plasticity is believed to be important in the adult brain as well.

The recruitment of AMPA receptors to synapses may occur by two mechanisms, 1) the synthesis of the receptor in the soma and transport to the dendrites, or 2) targeting of dendritically synthesized glutamate receptors to specific synapses. Transport of glutamate receptors from the soma to the dendrite is known to occur. However, there is no evidence that dendritic translation of glutamate receptor mRNAs also contributes to the dendritic population of receptors. Recent studies have shown that many mRNAs are present in neuronal cells (Miyashiro et al. 1994. Proc. Natl. Acad. Sci. USA 91:10800-10804; Crino, P. and J. Eberwine. 1996. Neuron 17:1173-1187). Among the mRNAs detected in dendrites are those that encode neurotransmitter receptors including the ionotropic glutamate (Miyashiro et al. 1994. Proc. Natl. Acad. Sci. USA 91:10800-10804) and GABA-A receptors (Crino, P. and J. Eberwine. 1996. Neuron 17:1173-1187) as well as Ca⁺⁺ channels (Crino, P. and J. Eberwine. 1996. Neuron 17:1173-1187). Glutamate receptors are integral membrane proteins that bind to glutamate and provide the primary excitatory responsiveness of the central nervous system. Translation of mRNAs encoding integral membrane proteins occurs on rough endoplasmic reticulum (RER) and the nascent proteins are then transported through the Golgi apparatus where they are modified and subsequently inserted into the membrane of the cell.

It has now been found that mRNAs encoding integral membrane proteins, such as neurotransmitter receptors, are locally translated in dendrites and then integrated into the neuronal cell membrane. This event occurs in response to various neurotransmitters and the fusion proteins formed are then integrated into the plasma membrane such that their distribution is indistinguishable from that of endogenous receptor proteins. Accordingly, neurotransmitter populations in neurons can be effectively modulated by altering mRNA translation of integral membrane proteins in dendrites.

### SUMMARY OF THE INVENTION

An object of the present invention is to provide compositions which modulate dendritic localized synthesis of neurotransmitter receptor populations in neurons which comprise an agent which alters mRNA translation of neurotransmitter receptors in dendrites of neurons.

Another object of the present invention is to provide a method of modulating neurotransmitter receptor populations of neurons comprising contacting dendrites of neurons with an agent which alters mRNA translation of neurotransmitter receptors in the dendrite.

### DETAILED DESCRIPTION OF THE INVENTION

A method for determining mRNA expression profiles in isolated cells was described in U.S. Pat. No. 5,723,290. This method also allows for profiling of mRNA expression in discrete segments of the same neuron. It has now been found that mRNAs of neurotransmitters, shown to be present in profiling, are synthesized locally in dendrites and inserted into the cell membrane. Thus, it is believed that dendrite localized synthesis via mRNA translation effects the levels of receptor populations in neurons, and ultimately the excitability of the neuron. Accordingly, neurotransmitter receptor populations can be modulated by altering synthesis of these receptors in dendrites. The ability to modulate neurotransmitter receptor populations is believed to have important implications for many diseases and neuropsychiatric conditions of the brain and central nervous system.

In the present invention “modulation” refers to the increase or decrease in the number of receptors in the population in a membrane of a cell. The cells of interest in the present invention are neuronal cells of animals, including humans.

Primary dissociated neuronal cultures were generated from embryonic rat hippocampi as previously described (Buchhalter, J. and M. Dichter. 1991. Brain Res. Bull. 26:333-342) and grown on microgrid coverslips (Eppendorf, Hamburg, Germany). Cell bodies of hippocampal neurons at 72 hours or 96 hours in culture were severed from their dendrites with a microelectrode and then removed by aspiration, leaving the isolated dendrites adhered to the coverslip. Photomicrographs were taken and used to locate the transfected dendrites for light and electron microscopic analysis. Isolated dendrites were transfected with glutamate receptor subunit 2 (GluR2) mRNA tagged at the c-terminus of the protein coding region with a mRNA sequence encoding a c-myc epitope (Crino, P. and J. Eberwine. 1996. Neuron 17:1173-1187). GluR2 c-myc capped RNA (10 μg) along with carrier tRNA (10 μg) was allowed to complex with 10 μg cationic DOSPER Liposomal Transfection Reagent (1,3-di-oleoyloxy-2(6-carboxy-spermy)-propylamide; Boerhinger-Mannheim) for 15 minutes at room temperature. The lipid/mRNA complex was applied directly onto severed dendrites with a microelectrode. Protein synthesis was stimulated by incubation of the cultured dendrites with the neurotrophic factor BDNF for 2 hours or the metabolic glutamate receptor agonist (RS)-3-5-dihydroxyphenylglycine (DHPG) for 30 minutes. Following RNA transfection, cells were washed and fixed in 4% paraformaldehyde, incubated in 5% normal goat serum with 0.1% Triton-X-100 and labeled with anti-c-myc monoclonal antibody, overnight at 4 [symbol] C. The avidin-biotin conjugation method was used to process the monoclonal antibody (Vectastain ABC, Vector Labs). 3,3-diaminobenzidine was used to visualize immunoreactivity. Local translation of the transfected mRNAs was assessed by light microscopy of the immunohistochemically amplified c-myc epitope tag present in the glutamate receptor mRNA. This protein synthesis assay was first utilized to prove that protein synthesis can occur in isolated dendrites (Crino, P. and J. Eberwine. 1996. J. Neuron 17:1173-1187). Following transfection of lipid-mRNA complex and DHPG treatment, c-myc immunoreactivity was visible in intact neurons as well as in single isolated dendrites. Similar results were found in c-myc fusion construct mRNAs of GluR4 and NMDAR1. No immunoreactivity was detectable without transfection of lipid-mRNA.

Double-labeling immunogold localization of c-myc in combination with NMDAR1 of GluR2 was used to validate the light microscopic evidence that translation is occurring in the transfected dendrites and to determine whether or not locally synthesized glutamate receptor molecules are present at the plasma membrane. In order to apply immunogold electron microscopic techniques to the preparation, essential elements of two procedures were used with modifications for cell culture preparations. Coverslips containing transfected dendrites were fixed with 2.5% glutaraldehyde, 1% paraformaldehyde, and 0.1% picric acid in 0.1 M phosphate buffered saline (PBS, pH 7.3) for 30 minutes at 4 C, washed with 0.1 M PBS and transferred to 0.1 M maleate buffer (MB, pH 6.0).

Tissue embedding was processed. Tissue was treated with 1% tannic acid/MB, 0.1% CaCl₂/MB, 1% uranyl acetate (UA)/MB, and 0.5% platinum chloride/MB, and dehydrated through a graded ethanol series up to 70% ethanol, treated with 1% para-phenyldiamine/70% ethanol, and followed with 1% UA/70% ethanol. Dehydration was carried up to 100% ethanol, followed by propylene oxide and infiltration with Araldite resin. An inverted Beem capsule which had the bottom cut off was placed over the area which contained the transfected dendrites and a few drops of resin were added to cover the dendrites. After polymerization for two to three hours, the entire Beem capsule was filled and polymerization was completed at 50 [symbol] C for 36 hours. The Beem capsule and material were separated by liquid nitrogen immersion. Photomicrographs were used to identify the transfected dendrites and the immunogold procedure was initiated.

Ultrathin sections were cut by diamond knife on a Reichert-Jung ultramicrotome and thin sections were collected on uncoated nickel grid (300 mesh) treated with Quick Coat (EMS, Fort Washington, Pa.). Grids were transferred to grid holder plates, rinsed in Tris-buffered saline containing 0.1% Triton-X 100 (TBST) and incubated in the following solutions at room temperature: 1 minute in 0.1% sodium borohydride and 50 mM glycine/TBST, and for 10 minutes in TBST containing 2% human serum albumin. For single immunolabeling studies, sections were incubated with monoclonal anti-c-myc at 0.9 μg/ml, polyclonal anti-GluR2 (Chemicon, Temecula, Calif.) at 2.5 μg/ml, or polyclonal anti-NMDAR1 (Chemicon, Temecula, Calif.) at 2.5 μg/ml in the above diluent overnight at room temperature in a covered humid chamber. Sections were washed with TBST and incubated overnight for 1 hour at room temperature in a secondary gold tagged antibody in TBST (2% human serum albumin and polyethylene glycol 20,000; 5 mg/ml). Monoclonal anti-c-myc was incubated in goat anti-mouse IgG conjugated to 10 nm gold particles and the polyclonal anti-GluR2 or anti-NMDAR1 were incubated in goat anti-rabbit IgG conjugated to 15 nm gold particles. Double label post-embedded immunogold studies were performed using simultaneous incubation of the primary antibodies at the dilutions described above, followed first by a 1 hour incubation with a secondary antibody for the monoclonal antibody (1:40, goat anti-mouse IgG conjugated to 10 nm gold particles) and second by a 1 hour incubation in a secondary antibody for the polyclonal antibody (1:40. goat anti-rabbit IgG conjugated to 15 nm gold particles). In independent control experiments the primary antibody was excluded before incubation with gold-tagged secondary antibodies. Grids were washed and stained with uranyl acetate and lead citrate and examined at 80 kV on a Joel 1200EX electron microscope.

In both the GluR2/c-myc double-labeled material and the NMDAR1/c-myc double-labeled material c-myc labeling was pronounced in the dendrites proving that the GluR/c-myc fusion construct mRNA was successfully translated locally into the GluR with a c-myc tag. Co-localization studies with NMDAR1 demonstrated that both c-myc and NMDAR1 were present in the same dendrite and often in close proximity to one another. The GluR2/c-myc labeled material was particularly illuminating in that there were several examples of GluR2 and c-myc immunogold localization in very close proximity to each other within the plasma membrane. The gold particles were within the theoretical limit of lateral resolution of the technique used suggesting that either endogenous GluR2 and exogenous GluR2/c-myc molecules were present in the same AMPA receptor complex or that the GluR2 labeling and the c-myc labeling represented binding to each respective epitope on the same protein molecule that was translated from the endogenous mRNA. It is unlikely that two antibodies were reacting with the same protein molecule because that would have led to many more closely packed immunogold particles. Regardless of the mechanism, these data demonstrate that locally synthesized GluR2 was inserted in the membrane and would be capable of forming complexes with GluR2 molecules.

Although the presence of rough endoplasmic reticulum and Golgi apparatus in dendrites has been controversial, the demonstration that integral membrane proteins are synthesized in dendrites and inserted into the membrane indicates that there is functional rough endoplasmic reticulum and Golgi apparatus in the dendrites. The demonstration that synthesis and membrane insertion of integral membrane proteins occurs in the dendrite indicates that the glutamate responsiveness of synapses is linked to the pool of neurotransmitters in the synaptic cleft. Further, the local synthesis and membrane insertion of glutamate receptors in the dendrites indicates there is a role for the conversion of silent to active synapses as well as other dynamic shifts in neuronal functioning that would follow.

Based upon the experiments, it is believed that neurotransmitter receptor populations in neurons, such as glutamate receptors, can be modulated by altering translation of dendritically localized mRNAs. mRNA translation can be altered via agents well known in the art such as antisense oligonucleotides or ribozymes linked to antisense oligonucleotides which are specifically targeted to select mRNAs. Thus, compositions comprising such an agent can be used to alter translation of selected mRNAs in dendrites thereby modulating the neurotransmitter receptor population of the neurons. Accordingly, the present invention relates to both compositions and methods of using compositions comprising an agent which alters translation of selected mRNAs in dendrites to modulate neurotransmitter populations in neurons. In one embodiment, the agent comprises an antisense oligonucleotide which hybridizes to selected mRNAs, thereby inhibiting translation of the mRNA and decreasing the population of selected neurotransmitter receptors in the neuron. In a preferred embodiment, the antisense hybridizes to mRNA encoding glutamate receptors. In another embodiment, the agent comprises a metabolic receptor agonist such as (RS)-3-5-dihydroxyphenylglycine (DHPG), which, as demonstrated herein, increases glutamate receptor populations.

What is claimed is:

1. A composition for decreasing glutamate receptor numbers in dendrites of neurons comprising an antisense oligonucleotide, or a ribozyme linked to an antisense oligonucleotide, wherein said antisense oligonucleotide is specifically targeted to a dendrite-localized mRNA that encodes for said glutamate receptor which decreases mRNA translation of said glutamate receptors in dendrites of neurons.
